(19) United States
(12) Plant Patent Application Publication
(10) Pub. No.: US 2015/0296694 P1
(43) Pub. Date: Oct. 15, 2015

(54) Variety of Calla Lily Plant Named 'Gran Paradiso'

(50) ;

(71) Applicant: Sande B.V., 't Zand (NL)

(72) Inventor: Cecilius Jan-Jochem Randag, 't Zand (NL)

(21) Appl. No.: 14/544,487

(22) Filed: Jan. 12, 2015

Related U.S. Application Data
(60) Provisional application No. 61/964,771, filed on Apr. 11, 2014.

(51) Int. Cl. A01H 5/00

(52) U.S. Cl. PLT/490

(57) ABSTRACT

‘Gran Paradiso’ is a new variety of calla lily having flowers with an orange red-colored spathe that produces 3-10 flowers per tuber. The flower stems are about 90 cm in height and the leaves are green with whitish spots and have a leathery texture.

### CROSS REFERENCE TO RELATED APPLICATION

This application claims the benefit of U.S. Provisional Patent Application Ser. No. 61/964,771, having a filing date of Apr. 11, 2014, and entitled “Varieties of Calla Lily Plants”, the entire disclosure of which is incorporated herein by reference.

### BOTANICAL CLASSIFICATION

Zantedeschia sprengeri

### VARIETAL DENOMINATION

‘GRAN PARADISO’

### BACKGROUND OF THE NEW PLANT

The present invention comprises a new and distinct cultivar of calla lily, botanically known as Zantedeschia sprengeri and hereinafter referred to by the cultivar name ‘Gran Paradiso’.

‘Gran Paradiso’ is a product of a planned breeding program which had the objective of creating Zantedeschia hybrids for cut flower production in a wide range of colors with a large, classic flower shape. The breeding program began in 1989 and the new cultivar is a seedling selected from the crossing of Zantedeschia sprengeri seedling Z030624 (female parent, unpatented) with Zantedeschia sprengeri variety ‘Treasure’ (male parent, unpatented). ‘Gran Paradiso’ was selected in 2008 by the inventor C. J. J. Randag in 't Zand, The Netherlands as one flowering plant within the progeny of the stated cross.

The first act of asexual reproduction of ‘Gran Paradiso’ by tissue culture was performed by the inventor in August of 2008 in 't Zand, The Netherlands. Further asexual reproductions by tissue culture at the same location has demonstrated that the combination of characteristics as herein disclosed for the new cultivar are retained and reproduced true to type through successive generations of asexual reproduction.

The following observations, measurements and comparisons describe plants grown in 't Zand, The Netherlands under greenhouse conditions which approximate those generally used in horticultural practice. Color references are made to the R.H.S. Colour Chart of The Royal Horticulture Society of London, except where general color terms of ordinary significance are used.

The present invention has not been evaluated under all possible environmental conditions. The phenotype may vary with variation in environment without a change in the genotype of the plant.

The following traits have been repeatedly observed and determined to basic characteristics of ‘Gran Paradiso’ which, in combination, distinguish this calla lily as a new and distinct cultivar:
1. Orange red-colored spathe;
2. Big spathe size;
3. Tall inflorescence stems; and
4. High inflorescence production.

Table 1 provides similarities and differences of ‘Gran Paradiso’ to its parents, Z030624 and ‘Treasure’.

TABLE 1

| ‘Gran Paradiso’ | Similarities | Differences |
|---|---|---|
| Z030624 | Leaf shape | Number of spathes |
| | Spathe color | Spots on leaves |
| ‘Treasure’ | Leaf shape | Number of spathes |
| | | Spathe shape |

### BRIEF DESCRIPTION OF THE DRAWINGS

The accompanying photographic drawings illustrate the new variety, with the colors being as nearly true as is possible with color illustrations of this type:

FIG. 1 is a photograph of a single plant of the new variety; and

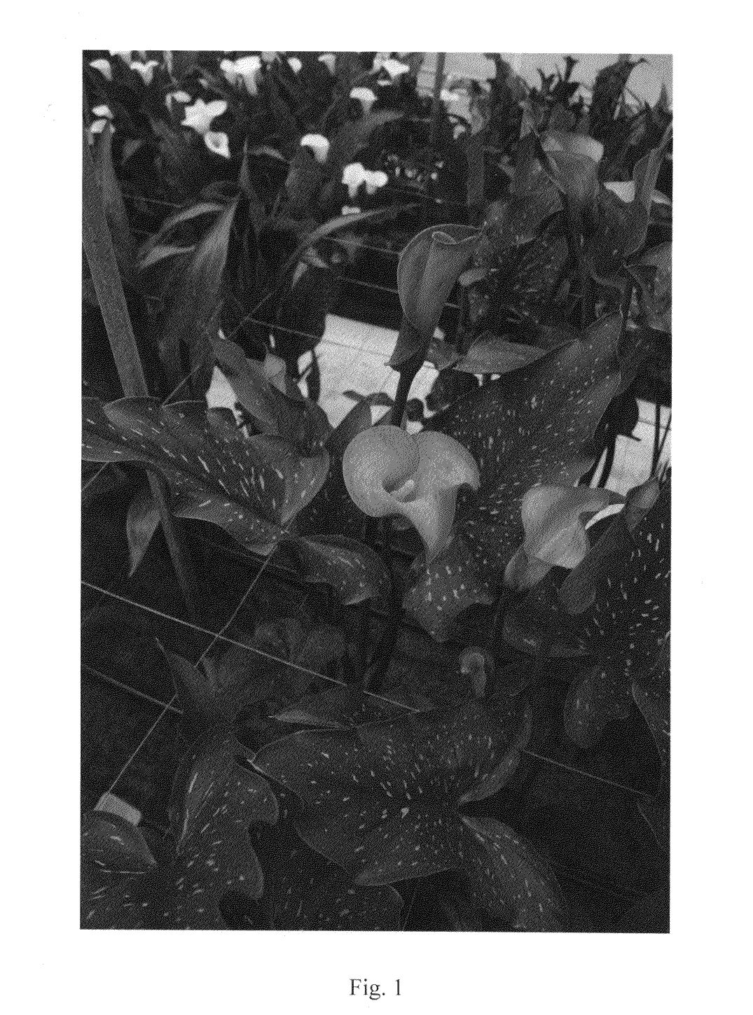

FIG. 2 is a photograph of a group of multiple plants of the new variety.

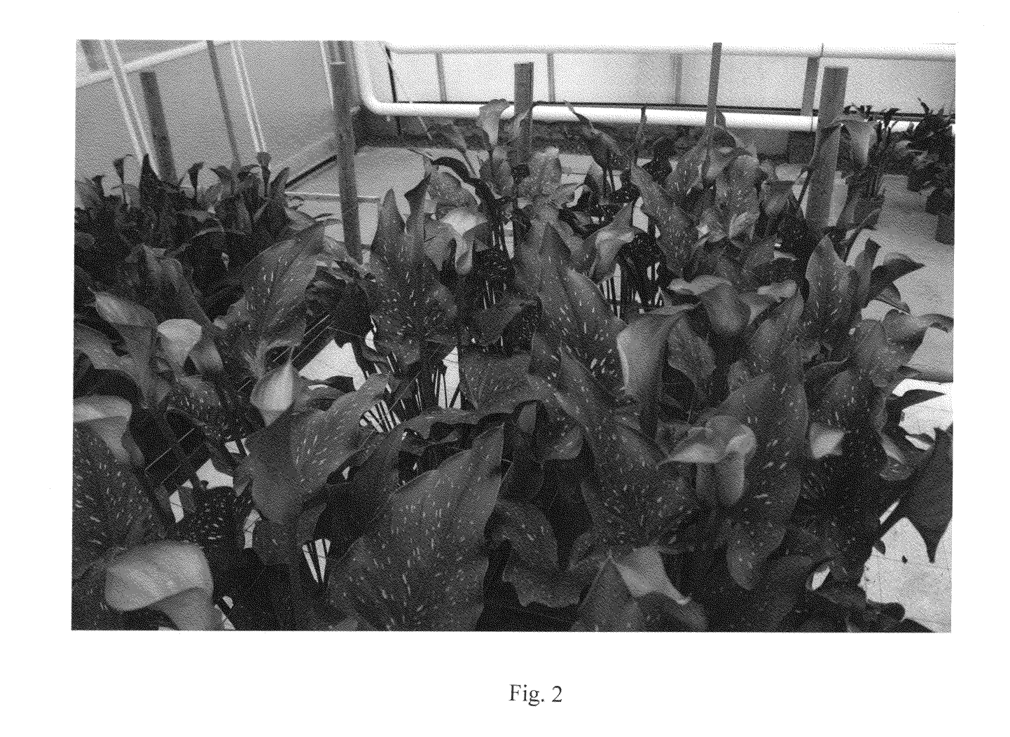

### DESCRIPTION OF THE PLANT

The plant:
Size:
   Height of the leaf canopy above the soil.—25-95 cm.
   Height of top of inflorescence above the soil.—50-100 cm.
   Diameter.—25-60 cm.
Form: Erect.
Number of inflorescences per tuber:
   Size 14-16 cm in diameter.—3-6 inflorescences.
   Size 16-18 cm in diameter.—5-8 inflorescences.
   Size 18-20 cm in diameter.—6-10 inflorescences.
Branches:
   Character.—Like the variety ‘Picasso’ (U.S. Plant Pat. No 15,282).
   Color.—Between Yellow-Green Group RHS 147A and 147B.
Leaves:
   Size.—Width: 5-25 cm. Length: 7-30 cm.
   Number per plant.—7-20.
   Shape.—Sagittate.
   Color.—Upper surface: Green Group RHS 137A to 137B. Lower surface: Green Group RHS 138A to 138B.
   Margin.—Undulate, having a color between Green Group RHS 137A to 137B.
   Veins.—Configuration: Pinnate. Color: Green Group RHS 138A to 138B.
   Surface quality.—Leathery.
   Petiole.—Length: 20-70 cm. Color: The base is White Group RHS 155A, and the upper part of the petiole is between Green Group RHS 138A and 138B.
Roots:
   Color.—White.
   Branching.—Similar to Zantedeschia varieties.
The flower and seeds:
Spathe:
   Size.—Length (measured from base opening to tip): 4.5-10 cm. Width: 3-8 cm. Height 7-14 cm.
   Color.—Upper surface: Varies between Orange Group RHS 25A and 25D. Lower surface: Varies between Yellow-Green Group RHS 144A and 144B.
   Vein color.—Varies between Orange-Red Group RHS N34A and N34B.
   Shape.—Cupped.
Spadix:
   Size.—Length: 30-70 mm. Diameter: 3-13 mm.
   Color.—Between Yellow-Orange Group RHS 23A and 23B.
   Position relative to spathe.—Upright.
Peduncle:
   Size.—Length: 40-90 cm. Diameter: 3-18 mm.
   Color.—Upper part varies between Green Group RHS 137A and 137B, and the lower part is Green-White Group RHS 157C.
Reproductive organs:
   Location of female organs.—Basal position of the spadix.
   Location of male organs.—Upper position of the spadix.
   Perianth.—Conspicuous.
   Stamens.—Presence: Not visible before pollen release. Number: More than 50. Pollen amount: Similar to ‘Picasso’ (U.S. Plant Pat. No. 15,282). Color: Between White Group RHS 155A and 155B.
   Anther.—Shape: Round and wire-like. Length: Less than 1 mm. Color: Between Yellow-Orange Group RHS 23A and 23B.
   Filament.—Length: Less than 1 mm. Color: Between White Group RHS 155A and 155B.
   Pistil.—Number: 30 to 50. Length beyond perianth: About 1 mm.
   Stigma.—Shape: Round. Size: Less than 1 mm.
   Style.—Length: Less than 1 mm. Color: Between Yellow-Orange Group RHS 15A and 15B.
   Ovaries.—Texture: Smooth. Length: Between 1 to 10 mm. Width: Between 1 to 10 mm. Color: Top color varies between Green Group RHS 140B and Orange-Red Group N34A, bottom color varies between White Group RHS 155B and Green-White Group 157C.
Seeds:
   Length.—4-7 mm.
   Width.—3-6 mm.
   Color.—Between Yellow-Green Group RHS 150A and 151A.
   Amount.—Between 1-100.
General:
Disease resistance: No unusual susceptibility to diseases noticed to date.
Pest resistance: No unusual susceptibility to pests noticed to date.
Heat tolerance: No heat tolerance.
Hardiness: Not hardy.
Flowering: Tubers planted in a greenhouse in The Netherlands in February produce flowers in April. Tubers planted outdoors in The Netherlands in May produce flowers in July.
Lastingness: About two weeks on the plant.
Fragrance: None.

I claim:

1. A new and distinct variety of calla lily plant named ‘Gran Paradiso’ as herein described and illustrated.
